(19) United States
(12) Patent Application Publication
(10) Pub. No.: US 2009/0233350 A1
(43) Pub. Date: Sep. 17, 2009

(54) RESPIRATORY APPARATUS WITH A BIOBURDEN INDICATOR

(75) Inventors: Camillo BELLUZZI, Ostiglia (MN) (IT); Sarah Gallini, Finale Emilia (IT); Massimiliano Solci, Correggio (RE) (IT)

Correspondence Address: CARTER, DELUCA, FARRELL & SCHMIDT, LLP, 445 BROAD HOLLOW ROAD, SUITE 420, MELVILLE, NY, 11747, US

(73) Assignee: Covidien AG, Neuhausen am Rheinfall (CH)

(21) Appl. No.: 12/400,947

(22) Filed: Mar. 10, 2009

(30) Foreign Application Priority Data
Mar. 14, 2008 (EP) 08425159.4

(51) Int. Cl. C12M 1/00; G01N 21/00

(52) U.S. Cl. 435/288.7; 422/85; 435/287.1

(57) ABSTRACT

A respiratory apparatus includes a bioburden indicator, having an input valve for the entry of air to be analysed, an element for bioburden analysis, and a suction valve suited to be connected to a suction system to suck up the air to be analysed.

### TECHNICAL FIELD

The present disclosure concerns a respiratory apparatus with a bioburden indicator.

### BACKGROUND

At present, respiratory devices, such as those used for mechanical ventilation in anaesthesia departments and intensive care units (ICU), do not allow the user to assess rapidly and effectively the presence of microorganisms in the respiratory device itself. Clearly, this exposes the patient to a possible risk of infection, the consequences of which are not always foreseeable.

For a correct assessment of this problem, it must be noted that the environments in which the respiratory devices considered are used are hospitals or similar environments, and therefore places with a high risk of infection. Moreover these devices are used on patients whose immune defense system is frequently impaired and are more endangered by any infection, caused both by pathogen and opportunist organisms.

The object of the present disclosure is to realise a bioburden indicator which allows the user to assess rapidly and effectively the presence of bioburden in a respiratory apparatus.

### SUMMARY

The present disclosure concerns a respiratory apparatus having a bioburden indicator which includes an input valve for the entry of air to be analysed, an element for bioburden analysis, and a suction valve suited to be connected to a suction system to suck up the air to be analysed.

### BRIEF DESCRIPTION OF THE DRAWINGS

Various embodiments of the present disclosure will be described herein below with reference to the figures wherein:

FIG. 1 illustrates a bioburden indicator according to the present disclosure and connected to a respiratory branch of a respiratory apparatus; and

FIG. 2 shows an exploded view of the bioburden indicator illustrated in FIG. 1.

### DETAILED DESCRIPTION

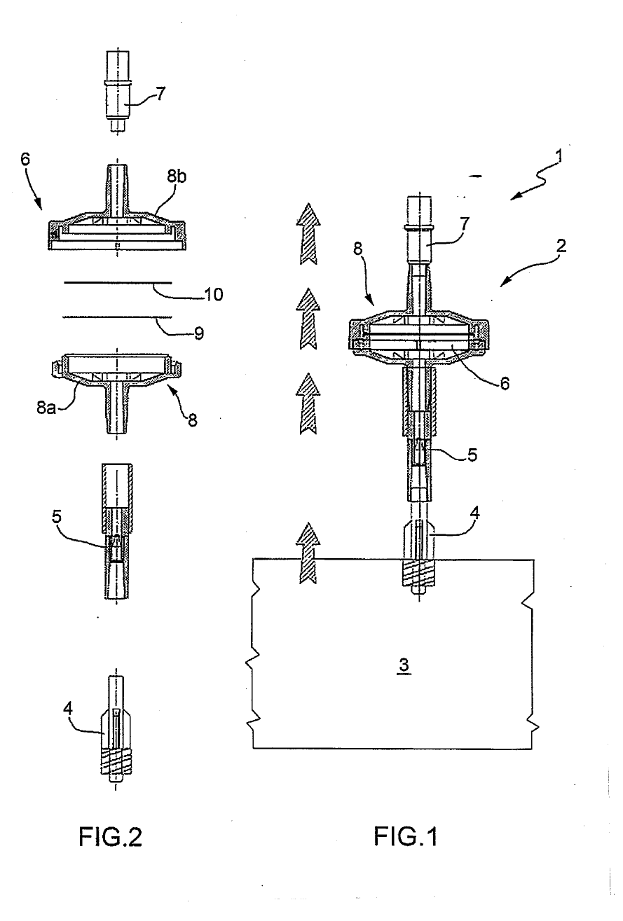

In FIG. 1, reference 1 indicates overall a respiratory apparatus, which is only partially illustrated. The respiratory apparatus 1 includes a respiratory branch 3 that may be utilized, for example, as a tube in an ICU ventilation system or an expiration hose of a breathing filter, a bioburden indicator 2 and a Luer connector 4 suited to connect together the bioburden indicator 2 and the respiratory branch 3.

The bioburden indicator 2 may include, in assembly sequence, a unidirectional input valve 5 fitted on the Luer connector 4, a bioburden analysis element 6 suited to supply a visual response of the microbial burden presence, and a suction valve 7 connected to the bioburden analysis element 6 on the side opposite the input valve 5. The bioburden analysis element 6 may include a colorimetric element suited to change colour when a determined level of bioburden is reached.

In use, the suction valve 7 may be coupled to a suction system, for example a syringe, to suck up the air from the respiratory branch 3.

As illustrated in FIG. 2, the analysis element 6 may include a containing structure 8 including a bottom part 8a and a top part 8b which may be connected together, an analysis disk 9, and a bacterial/viral filter 10 located between the analysis disk 9 and the suction valve 7, that is downstream from the analysis disk 9 with respect to the flow of air to be analysed.

The analysis disk 9 may include a bioburden growth medium capable of favouring the growth of any microbial burden present in the air to be analysed and to change colour when a determined level of microbial burden is reached. For this purpose, the containing structure 8 may be made of a transparent material, or have at least a transparent window, so that the user can see when the analysis disk 9 changes colour.

In use, the Luer connector 4 may be connected to the respiratory branch 3 by a screw connection to any sampling port, and the bioburden indicator 2 may be coupled to the Luer connector 4 by a male/female coupling on the unidirectional input valve 5. Once the working conformation has been implemented as above, the suction valve 7 may be connected to a suction system, by the action of which air is sucked up from the respiratory branch 3 to be brought in contact with the analysis disk 9. The unidirectional input valve 5 may be configured to allow the analysis element 6 to be effectively isolated from the patient.

A change in colour then takes place on the analysis disk 9 depending on whether or not the microorganisms presence has exceeded a determined level of colonisation. This change will inform the user of the need to change at least the impaired parts of the respiratory device due to the high presence of bioburden.

The opportunely sealed bacterial/viral filter 10 may be configured to prevent the bioburden present in the analysis disk 9 from escaping from the suction valve 7, resulting in the safety of the operator and of the environment.

As may appear immediate from the above description, the respiratory apparatus of the present disclosure offers the notable advantage of being able to know immediately the extent of bioburden presence inside respiratory branches, without this involving any type of risk for the patient or for the operator.

1. A respiratory apparatus comprising:
a bioburden indicator; the bioburden indicator including
an input valve for the entry of air to be analysed,
a bioburden analysis element, and
a suction valve suited to be connected to a suction system to suck up the air to be analysed.

2. A respiratory apparatus according to claim 1, wherein the bioburden analysis element includes a colorimetric element able to change colour when a determined level of bioburden is reached.

3. A respiratory apparatus according to claim 1, wherein the bioburden analysis element includes an analysis disk and a bacterial/viral filter located between the analysis disk and the suction valve.

4. A respiratory apparatus according to claim 3, wherein the analysis disk includes a microbial burden growth medium able to favour the growth of any microbial burden present in the air to be analysed.

5. A respiratory apparatus according to claim 2, wherein the bioburden analysis element includes a containing structure having at least one transparent window for viewing the change of colour.

6. A bioburden indicator suited to be connected to a respiratory branch of a respiratory apparatus comprising:
a unidirectional input valve for the entry of air to be analysed coming from the respiratory branch,
a bioburden analysis element including a colorimetric element suited to change colour when a determined level of bioburden is reached, and
a suction valve suited to be connected to a suction system to suck up the air to be analysed from the respiratory branch.

7. A bioburden indicator according to claim 6, wherein the bioburden analysis element includes an analysis disk and a bacterial/viral filter located between the analysis disk and the suction valve.

8. A bioburden indicator according to claim 7, wherein the analysis disk includes a bioburden growth medium able to favour the growth of any microbial burden present in the air to be analysed.

9. A bioburden indicator according to claim 6, wherein the bioburden analysis element includes a containing structure having at least one transparent window for viewing the change of colour.
